(19) United States
(12) Patent Application Publication
(10) Pub. No.: US 2021/0330912 A1
(43) Pub. Date: Oct. 28, 2021

(54) FACE MASKS WITH FILTERS, FACE PLATE FOR USE WITH FACE MASKS, AND TREATMENT METHODS

(71) Applicant: Groman Inc., Margate, FL (US)

(72) Inventors: Boaz Barry Groman, Boca Raton, FL (US); Paul Pesach Louis, Boca Raton, FL (US)

(21) Appl. No.: 17/239,735

(22) Filed: Apr. 26, 2021

Related U.S. Application Data
(60) Provisional application No. 63/154,674, filed on Feb. 27, 2021.
(60) Provisional application No. 63/059,170, filed on Jul. 31, 2020.
(60) Provisional application No. 63/019,979, filed on May 4, 2020.
(60) Provisional application No. 63/015,464, filed on Apr. 24, 2020.

(51) Int. Cl. A61M 16/00; A61M 16/06; A61M 16/20

(52) U.S. Cl. CPC A61M 16/0093; A61M 16/0622; A61M 16/0694; A61M 2202/0208; A61M 2205/7509; A61M 2205/7518; A61M 16/208

(57) ABSTRACT

An adapter for retrofitting filters to face masks. Filters welded to the mask. A spacer allowing a check valve to operate. A method of providing oxygen and/or a nebulizing treatment to a patient is disclosed. Modifications to a mask may include: adding nose cushion to increase conformance of the mask to facial features; covering breathing ports with viral/bacterial filters; and adding desiccant material to capture moisture accumulation. A face plate fitted over and working in conjunction with a mask which pushes the mask against a user's facial features to assure a better fit. Straps are disposed on the face plate, rather than on the mask. The face plate may provide or comprise: filter protection from user contact, strap locking features, and a nebulizer cutout.

### FIELD OF THE INVENTION

This invention relates to medical facemasks and filtering of air being breathed by a patient, particularly air which is exhaled by the patient, and which may contain undesirable aerosols and/or particulate matter (including suspensions).

This invention also relates to methods of providing low pressure oxygen and/or nebulizing treatments to a patient.

### BACKGROUND

In the 2020 Coronavirus pandemic, treatment options for patients with shortness of breath were limited due to the fear that first line and hospital personnel would be exposed to aerosolized viral particles expelled by patients into the environment.

Typically, for shortness of breath, the first line of treatment is oxygen. However, it soon became apparent, when treating patients with SARS-CoV-2, that oxygen via a nasal cannula at greater than six liters per minute, aerosolized viral particles and exposed personnel caring for the patient. In addition, patients with asthma and chronic obstructive pulmonary disease could not be treated with nebulized medications, the mainstay of these diseases, for the same reason.

Due to recent infections, ERs have stopped providing breathing treatments for all illnesses (asthma, heart, OCPD, Etc.) due to aeration concerns. Instead, many more patients are being intubated so ventilated air can be controlled. Intubation is a substantially more invasive, risky, and costly procedure.

Some Patent, And Other References

U.S. Pat. No. 6,659,102 (2003-12-09; Sico) discloses an oxygen mask filter system for preventing the transmission of a disease from a patient to medical personnel. The oxygen mask filter system includes a face mask having an interior surface and an exterior surface, a plurality of vent apertures, a disk member movably attached to the exterior surface of the face mask about the vent apertures, and a filter member attached to the interior surface of the face mask for filtering gases prior to expulsion from the interior of the face mask to the exterior through the vent apertures.

U.S. Pat. No. 6,854,464 (2005-02-15; Mukaiyama et al) discloses a respiratory protection apparatus has a face body, and a right side and a left side of a vertical centerline of the body are respectively provided with filtering devices each of which houses an electrical fan unit therein. Without narrowing the field of the front vision, without increasing the front side weight, and without deteriorating the work efficiency and comfortable fitting, the apparatus ensures the air flow rate of more than 120 liters per minute. Moreover, the electrical fan unit may be detachable from the face body, so that the face body is easily washed and parts are readily replaced with new ones.

U.S. Pat. No. 6,854,464 is an active device with a built in fan—The device disclosed herein (by Groman, Louis) is passive where positive air flow is generated by the nebulizer or oxygen supply.

U.S. Pat. No. 7,559,323 (2009-07-14; Hacke et al; Respan Products Inc.) discloses a face mask assembly and method of assembling a face mask is provided for a patient that includes a face piece sized to fit over the patient's nose and mouth. The face mask assembly forms a mask chamber between the face piece and the patient's nose and mouth. An inhalation adapter is coupled to the face piece to deliver medication to the chamber. A filter housing is coupled to the face piece and includes a flange section that defines a passageway to connect the mask chamber and the flange section. A filter is positioned in the filter housing. A cover is coupled to the flange section and has an exhalation opening or vent to allow gases from the mask chamber to pass through the filter and escape from the passageway to the atmosphere.

U.S. Pat. No. 7,559,323 provides means for attaching a retrofit filter to a face mask. Their mounting solution is not good because the face mask material is very soft and the filter assembly can pop off, but it's a very similar scenario. The retrofit adapter disclosed herein (by Groman, Louis) provides better, more robust support for the filter cartridge, because it has both inner and outer pieces “capturing” the mask material at the exhaust opening.

U.S. Pat. No. 8,342,179 (2013-01-01; Hacke et al.) discloses A face mask assembly and method of assembling a face mask is provided for a patient that includes a face piece sized to fit over the patient's nose and mouth. The face mask assembly forms a mask chamber between the face piece and the patient's nose and mouth. An inhalation adapter is coupled to the face piece to deliver medication to the chamber. A filter housing is coupled to the face piece and includes a flange section that defines a passageway to connect the mask chamber and the flange section. A filter is positioned in the filter housing. A cover is coupled to the flange section and has an exhalation opening or vent to allow gases from the mask chamber to pass through the filter and escape from the passageway to the atmosphere. In a second embodiment, the filter housing is modified by providing a frame adjacent the bottom with a cross-shaped structure and a retainer button attached to the housing. A valve disc is provided between the retainer button and frame adapted to allow gases from the mask chamber around the valve disc towards the filter. When a patient inhales oxygen through the nasal portion, the valve disc moves towards the mask and partially covers a plurality of apertures to partially block additional air from entering the chamber. When a patient exhales contaminated oxygen, the valve disc moves towards the retainer button to allow a maximum amount of air through the apertures towards the filter.

U.S. Pat. No. 8,464,715 (2013-06-18; Flynn, Sr.) discloses a therapeutic face mask comprises a face-engaging portion and a single connector having a mask-engaging end and a single treatment-receiving end which has a single attachment mounting for detachably sealingly receiving a treatment attachment, such as an oxygen reservoir bag or a nebulizer. A one-way inhalation valve in the connector permits fluid flow from the treatment-receiving end to the mask-engaging end during inhalation and inhibits fluid flow in the other direction. The mask also includes a valve-governed exhalation port and an anti-asphyxia valve assembly configured to permit fluid flow from ambient to the face-engaging portion during inhalation only when inspiratory effort during inhalation exceeds fluid flow to the treatment-receiving end of the connector. Also provided is an oxygen reservoir bag having a neck shaped for removable coupling to a mating connector of a therapeutic face mask. An oxygen reservoir bag may have a metered-dose inhaler port defined in its neck.

U.S. Pat. No. 8,534,280 (2013-09-17; Dhuper et al) discloses a device for use in an aerosol inhalation system for delivering aerosolized medication includes a housing that is operatively connected to a source of aerosolized medication such that the aerosolized medication is delivered to the housing. The device also includes a patient interface member removably connected to the housing and being separate therefrom. The patient interface member is in the form of a face mask for placement about a face of the patient and in communication with a mouth of the patient for delivering the aerosolized medication. The patient interface member incorporates an integral inhalation valve and safety feature for protecting against displacement of the inhalation valve.

U.S. Ser. No. 10/335,569 (2019-07-02; Beard et al), discloses An oxygen face mask and component system is provided, the mask is designed to cover a user's nose and at least partially cover a user's mouth, the mask having lateral ports. Systems and assemblies including such a face mask and additional components are further provided, including a colorimetric CO2 detector, a sealing cap with or without a resilient sealing flap, a capnography gas analysis unit, a non-rebreather valve, a pulmonary function module, nebulizer, a gas scavenging system, a gas reservoir system, a gas filter, sample lines that are either straight or at an angle, and an aerosol mask platform; and methods of making and using such a face mask are also provided.

U.S. Ser. No. 10/576,313 (2020-03-03; Shigematsu et al) discloses a detachable connector between a face piece and a filter of a breathing apparatus is provided with a bayonet connection mechanism for connecting a pair of members by pushing and twisting operation, and a lock mechanism for locking a connection between the pair of members by fitting a pin in a hole provided in an elastic member, wherein connecting motion of the bayonet connection mechanism synchronizes with locking motion of the lock mechanism so that connection of the filter and locking of the connection are carried out simultaneously when the filter is connected to the face piece, while unlocking motion of the lock mechanism precedes disconnecting motion of the bayonet connection mechanism so that unlocking of the connection precedes disconnection of the filter when the filter is disconnected from the face piece.

D753816 (2016-04-12; Beard) discloses oxygen face mask with capnography monitoring ports.

US 20050028811 (2005-02-10; Nelson et al) discloses the multitask medical treatment respiratory apparatus may have a mask defining a chamber wherein the mask may have a first port, a second port and a venting valve. A reservoir bag having a first opening may be in communication with the first port or there may be a venturi device may be in communication with a second opening of the reservoir bag. One or more gas sources may be in communication with the venturi device. A medicant device may be mounted in the second port wherein the medicant device having a mouthpiece that may be inserted and retracted relative to the mask chamber and a patients mouth. The medicant device having a medicant chamber formed therein.

US 20130125896 (2013-05-23; Dwyer et al) discloses Side Plug-In Filter Cartridge. A respirator 10 that includes a mask body 14, a filter cartridge receptacle 15, and a filter cartridge 12. The filter cartridge 12 has a side 30 that is capable of being plugged into the receptacle 15. The filter cartridge 15 can be inserted into the proper position within the receptacle 15 while the mask body 14 is being donned. The securement can be achieved without having the user visibly witness the actual engagement. An audible click or other indication can be provided so that the user knows that proper engagement has been achieved. The engagement may exhibit little spacing between the filter cartridge 12 and the mask body 14, thereby improving viewer visibility and making the inventive respirator 10 more comfortable to wear.

US 20160184548 (2016-06-30; Wallnewitz et al) discloses BREATHING MASK WITH EMERGENCY BREATHING VALVE. A breathing mask has a mask body surrounding a cavity that is open towards one side and is intended for coming into contact with a human face around the mouth and/or nose. A valve arrangement is provided in the mask body with an inspiration valve, for controlling the flow of gas from a port for a breathing gas feed device for providing a breathing gas into the cavity, and an exhalation valve for controlling the flow of gas from the cavity into the surrounding atmosphere. An emergency breathing valve, controlling gas flow from the atmosphere surrounding the breathing mask into the cavity, has a pressure threshold, to allow gas flow, that is lower than an inhalation valve pressure threshold. The pressure thresholds at which the respective inhalation valve and the emergency breathing valve open are coordinated by the closing element being designed as a separate component of the valve arrangement.

The present invention discloses mounting a filter to the side vent of the mask, and the adapter disclosed herein, as well as the spacer disclosed herein, the face plate disclosed herein, and treatment procedures.

### SUMMARY

It is an object of the invention, in some of the embodiments disclosed herein, to provide improvements to medical facemasks, making them suitable for use with filters, such as with the adapter disclosed herein.

The broad concept of fitting a filter to a mask is known.

The present disclosure may provide some novel techniques for mounting the filter, along with other nuances not taught or disclosed in the prior art. This may include methods of mounting (such as welding) a filter permanently to the mask, with a spacer disposed between the filter and the mask to allow a check valve in the mask to function (e.g., deflect outwardly) unimpaired by the filter. This may also include using a separate faceplate to mount the mask on a patient's face, wherein the faceplate has its own mounting strap(s), thereby rendering mounting strap(s) on the mask superfluous, and the faceplate can help ensure a good (e.g., air-tight) seal between the mask and the patient's face.

It is an object of the invention, in some of the embodiments disclosed herein, to provide an improved method of performing nebulizer/breathing treatments, and the like, using a facemask with a filter. The filter may be retrofitted with a filter cartridge.

It is an object of the invention, in some of the embodiments disclosed herein, to provide an improved method of mounting a filter to a face mask, such as with the spacer disclosed herein.

It is an object of the invention, in some of the embodiments disclosed herein, to provide an improved method of mounting a facemask to a patient's face, using a separate instrumentality such as the face plate disclosed herein.

A simple oxygen mask type facemask covers a patients nose and mouth. It has an oxygen/nebulizer connection at the front. It has a ventilation hole on the side.

According to some aspects (or embodiments) of the invention(s) disclosed herein,
a conventional mask may be modified by providing a filter on its exhaust vent. The filter may be either (i) already integral with the mask, or (ii) retro-fitted to the exhaust vent of the mask with an adapter
a retrofit adapter allows a standard filter cartridge to be installed onto a conventional medical facemask.
with a nebulizer, providing the usual nebulizing treatment with a mask having a filter

According to the invention, generally, a mask of the type suitable for providing oxygen or nebulized (or aerosolized) treatment to a patient is fitted with a filter on its exhaust vent. This allows air exhaled be a patient to be filtered before it enters the environment. A spacer may be incorporated to allow a check valve to operate (open and close, freely). Treatment may include using a nebulizer with the mask which is modified to have a filter.

An adapter for retrofitting filters to face masks, and a spacer allowing a check valve space to operate. A method of providing oxygen and/or a nebulizing treatment to a patient is disclosed. Modifications to a mask may include: adding nose cushion to increase conformance of the mask to facial features; covering breathing ports with viral/bacterial filters; and adding desiccant material to capture moisture accumulation.

A face plate fitted over and working in conjunction with a mask which pushes the mask against a user's facial features to assure a better fit. Straps are disposed on the face plate, rather than on the mask. The face plate may provide or comprise: filter protection from user contact, strap locking features, and a nebulizer cutout.

According to some embodiments (examples) of the invention, an adapter for retrofitting filters (such as a standard filter cartridge) to face masks may comprise: an internal component sized to fit and be inserted through an opening in a wall of a face mask, to project from within the wall to outside of the wall; and an external component adapted to attach to a portion of the internal component projecting beyond the wall.

The internal component may have a hole extending therethrough to allow for the passage of air into or out of the mask, when it is mounted on the mask. The portion of the internal component projecting beyond the wall may be provided with means (such as external threads) for attaching with the external component. The external component may have a hole extending therethrough to allow for the passage of air into or out of the mask, when it is mounted on the mask. A first portion of the external component may be provided with means (such as internal threads) for attaching with the internal component. A second portion of the external component may be provided with means (such as external threads) for allowing a standard filter cartridge (having internal threads) to be mounted to the external component of the adapter, hence to be mounted to the facemask. The filter may subsequently be dismounted from the mask/adapter, but it is anticipated that both the mask and filter will be discarded (in an appropriate, safe manner) after use.

The external component may include a check valve for restricting air flow from inside the mask to outside the mask—i.e. to allow the patient's exhaled air to pass through the filter. A spacer may be incorporated to allow the check valve to move, and perform its intended function.

According to some embodiments (examples) of the invention, a method of providing oxygen and/or a nebulizing treatment to a patient may comprise: providing a mask fitting over a patient's mouth and nose, said mask comprising: (i) an opening (oxygen/nebulizer connection), typically located at the front of the mask, for admitting pressurized gas (oxygen) and/or a nebulized treatment, and (ii) a ventilation opening (vent) on a sidewall thereof for allowing ambient air to be inhaled by the patient and also allowing air exhaled by the patient to be exhausted to the environment; characterized by providing a filter on the vent.

An adapter may be provided for retrofitting the filter to the mask. Alternatively, the filter may be permanently (“integrally”) mounted to the mask, such as by welding or with an adhesive.

A check valve may be incorporated, either on the mask itself, or on the filter, or on the adapter, for allowing air exhaled by the patient to be expelled through the filter into the environment without allowing air to be inhaled by the patient through the filter and vent.

According to some embodiments (examples) of the invention, a face plate for securing a face mask to the face of a patient, may comprise: a structure having a periphery conforming to a periphery of the face mask, and suitable to be disposed over the mask when the mask is on the patient's face; and at least one strap extending from the structure for securing the face plate to the patient's face, thereby negating a need for a straps extending from the face mask.

The structure may be formed of a material that is more rigid than the material of the face mask. The structure may spread forces more evenly around the periphery of the mask, thereby enhancing a seal between the mask and the patient's face. The structure may have a cutout in an area of the patient's nose. The structure may have a cutout in an area of the patient's mouth, for nebulizer treatment.

The faceplate works in conjunction with a face mask, such as the various embodiments of face masks that may be disclosed herein.

The face plate is not fixed to the mask. It is adjusted to contact the patient's nose

The face plate may push the mask against a wearer's facial features.

A strap may be disposed on the face plate, rather than on the mask.

The face plate distributes the strap force onto the facial features—nose, cheeks, and chin.

The face plate may provide or comprise:
1. Filter protection from user contact
2. Strap locking features
3. Nebulizer cutout

A cutout may be provided on the bottom of the faceplate for easy mounting of a nebulizer device.

Strap locking features may be provided on the sides of the faceplate to provide enhanced holding pressure, and adjustability.

Filter protection surfaces may be provided on the face plate.

A method for using any nasal cannula End Tidal CO2 Capnography circuit with the mask.

Placing nasal cannula ports in the mask allows ET CO2 monitoring with no aerosol leakage. The nasal cannula ports are covered when not used.

The facemask may be modified to reduce aerosolization, and may have:
Nose Cushion to Increase Conformality to facial feature
Breathing ports covered with Viral/Bacterial filters
Desiccant material to capture moisture accumulation

A facemask with an exhaust valve, and a spacer disposed between the exhaust valve and a filter.

Generally, a retrofit adapter disclosed herein allows a standard filter cartridge to be installed onto a conventional medical facemask. The adapter may have an internal component disposed on an inside surface of a wall of the facemask at the location of a hole which is already there for allowing exhaled gases to be vented from the mask, and extends beyond the external surface of the mask where an external component may be joined thereto, sandwiching the facemask wall therebetween. The external component may be externally threaded to permit installation of a standard filter cartridge to the mask. A check valve (one way valve) may be incorporated into or onto the external component.

A filter may be fitted to the mask, over the check valve (exhaust valve). The valve may be a flap type valve, and in order to prevent the filter from impeding the function of the valve, a spacer may be installed over the valve, between the valve and the filter, to ensure adequate clearance for the check valve to operate.

Some methods of nebulizer/oxygen treatments, working in conjunction with the mask and accessories (filter adapter, spacer, face plate, etc.) may be disclosed herein.

A face plate may be provided which has its own straps for securing the face plate, and a mask supported by the face plate, to a patient's face (head). This eliminates the need for straps on the mask. The face plate may typically be relatively more rigid than the softer mask, and may ensure a more secure and air-tight fit of the mask on the patient's face.

According to some embodiments (examples) of the invention, a standard B/V filter may be modified (cut to a specific size and shape) so that it may be slightly larger than and fitted over exhaust opening(s) in an oxygen or nebulizer mask, and attached to the mask surface, using ultrasonic welding or an adhesive, to cover the opening(s). The spacer disclosed herein may be incorporated between the filter and the opening to allow a check valve in the opening to operate (i.e., to deflect to the “open” position). The face plate disclosed herein may be fitted over the mask to ensure an air-tight fit of the mask on the patient's face.

According to some embodiments (examples) of the invention, a nebulizer (or oxygen) type mask may comprise: a bacterial/viral (B/V) filter disposed on an opening in the mask. A peripheral portion of the B/V filter may be welded to a corresponding peripheral portion of the opening in the mask. There may be two openings on the mask, one on the left side of the mask, and one on the right side of the mask (corresponding to the left and right sides of the patient's face when the mask is worn). There may be two B/V filters, one B/V filter disposed over each of the two openings. A check valve may be disposed in the opening), for allowing a patient's exhaled air to be exhausted from within the mask, to the filter covering the opening in the mask. A spacer may be disposed between the check valve and the filter for allowing the check valve to operate (open), unimpaired by the filter. There may be a check valve, spacer, and filter on both sides (left, right) of the mask.

A separate face plate may be used with the mask for securing the mask to the face of a patient. the face plate may have its own straps for securely mounting the underlying mask to the patient's face, thereby negating the need for straps on the mask itself. The face place may comprise a structure having a peripheral portion conforming to a corresponding peripheral portion of the face mask, and suitable to be disposed over the mask when the mask is on the patient's face.

The mask with filter and separate face plate disclosed herein may constitute a “system”. The descriptions of some of the apparatuses disclosed herein may also set forth method of use, which may constitute methods of treating patients.

Other objects, features and advantages of the invention(s) disclosed herein may become apparent in light of the following illustrations and descriptions thereof.

### BRIEF DESCRIPTION OF THE DRAWINGS

Reference will be made in detail to embodiments of the disclosure, non-limiting examples of which may be illustrated in the accompanying drawing figures (FIGS.). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a photograph of a typical facemask for oxygen/nebulizer treatments, according to the prior art. The facemask may have openings (holes) in its wall surface.

FIG. 2 is a diagram (cross-sectional, exploded view) of an adapter for retrofitting filters to facemasks, according to an embodiment of the invention. The adapter may have an internal component and an external component.

FIG. 2A is a photograph of the internal component of the adapter shown in FIG. 2, inserted through a hole in a wall of the mask, according to an embodiment of the invention. A portion of the internal component of the adapter may project beyond the wall to be attached (connected, joined, mated) with the external component of the adapter. An external surface of the portion of the internal component which extends beyond the wall may be threaded (external threads).

FIG. 2B is a photograph of the external component of the adapter shown in FIG. 2, attaching to the internal component, according to an embodiment of the invention. The external component may be provided with internal threads to attach to (connect with) the external threads of the internal component. An external surface of the external component may be threaded (external threads) to accept a standard filter cartridge having internal threads.

The external component may attach to the internal component with threads, or with a “snap” (interference) fit to create a seal with the mask wall.

FIG. 2C is a photograph of a facemask with two filters mounted thereto, via the intermediary of two adapters (not visible) of the present invention.

FIG. 3 is a diagram of a mask with filter, and nebulizer, illustrating a method of treatment, according to an embodiment of the invention.

FIG. 4 is a diagram (cross-section, schematic) of the mask, spacer and filter, according to an embodiment of the invention.

FIG. 5 is a diagram (plan view) of a face plate for use with a face mask, according to an embodiment of the invention.

### DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

In an embodiment of the invention, generally, a filter retrofit for either an oxygen face mask or a nebulizer face mask which will allow a patient to receive either oxygen at high flow or nebulizers at high flow while filtering the environment from viral particles. This will allow the patients to receive initial or intermediate therapy such as high flow oxygen or nebulized medications. Filtered oxygen or filtered nebulized treatments can bridge patients to support their own airway and potentially either obviate or delay the extreme decision to intubate and place the patient on the ventilator.

The adapter disclosed herein is, in essence, a face mask filter “retrofit” to standard breathing treatment masks used in hospitals, ambulances, old age homes, etc. This retrofit is designed to capture/contain aeration during patient exhalation. The goal is not to create a 100% leak free mask, but rather to retrofit existing masks to reduce aeration exposure. This is also important to ambulatory services that provide first response and have to use existing masks until patient condition is assessed.

A spacer allowing a check valve to operate when a filter in installed on the face mask is disclosed and described.

Methods of performing nebulizer or oxygen treatments are disclosed and described.

A face plate for use in conjunction with a face mask is disclosed and described.

The various embodiments of the invention may be described in the Appendices, filed herewith, which generally disclose the following:

### APPENDICES

Appended hereto, and forming a part of the disclosure hereof are the following:

### Appendix 1: Face Mask Filter Retrofit Prototype Description

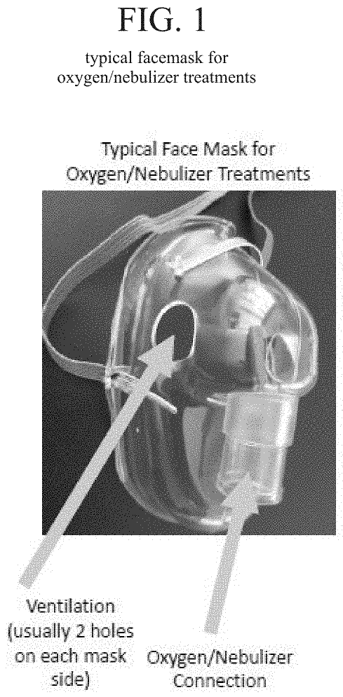

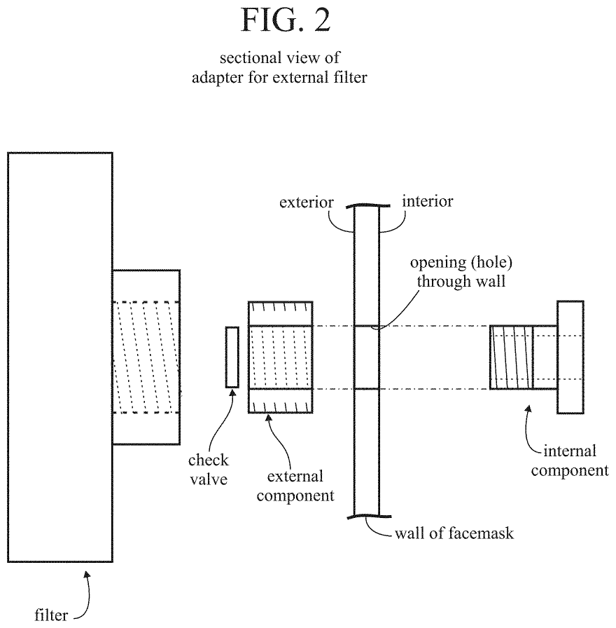

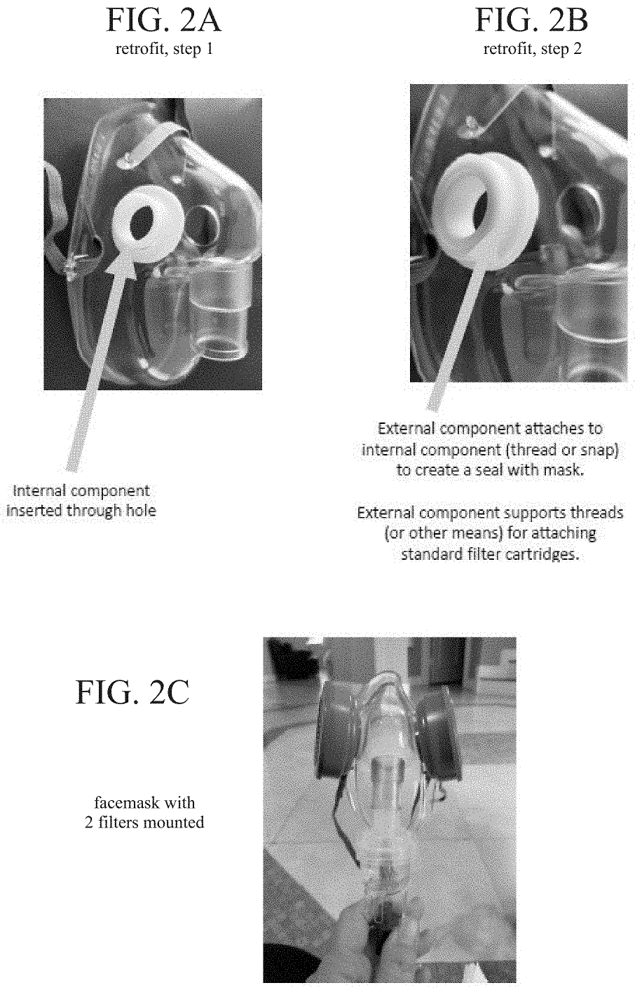

Some of the disclosure set forth herein may be described with reference to FIGS. 1, and 2A,B,C, and/or elsewhere in this specification.

### Appendix 2: Face Mask Filter Retrofit—Alternative Embodiments

Some of the disclosure set forth herein may be described elsewhere in this specification.

### Appendix 3: Current And Improved Nebulizer Methods

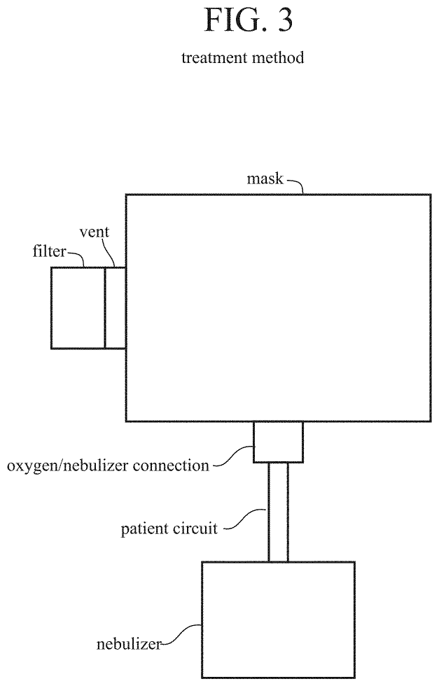

Some of the disclosure set forth herein may be described with reference to FIG. 3, and/or elsewhere in this specification.

### Appendix 4: Face Plate Embodiment

Some of the disclosure set forth herein may be described elsewhere in this specification.

### Appendix 5: Spacer

Some of the disclosure set forth herein may be described elsewhere in this specification.

### Appendix 6: Integrating(Welding) B/V Filters onto a Standard Nebulizer Mask

This appendix describes welding B/V (bacterial/viral) filters to a mask, rather than retrofitting canister filters to the mask. Given that the mask and filter should be disposable, this makes more sense.

Across the top, from left-to-right, the figures show:
this illustration shows a standard nebulizer mask below this is shown a B/V filter shaped to maximize surface area on Standard Nebulizer Mask
this illustration shows a Standard Nebulizer Mask with cutout (opening) of the filter shape, but slightly smaller perimeter to provide contact surface. (Both Mask Sides) In other words, the cutout should be shaped similarly to and slightly smaller than the filter.
this illustration shows a Filter is welded over the opening to create a maximum surface filtered opening. (Both Mask Sides)
to the right of this is shown a close-up of the Filter-Mask welded perimeter overlap
the illustration on the bottom right is an Image from the inside of the mask showing the welded filter over the mask cutout.

The filter may be a standard B/V filter, which may initially be round, cut/shaped to be a suitable size and shape for mounting onto cutouts in a mask, to which is it welded (adhered, using any suitable technique and/or adhesive)

This welded-on embodiment may be preferred over the “original” mask retrofit embodiment (adding a filter canister to the mask).

The mask with Viral-Bacterial filters welded to the mask is less expensive (filter canisters are costly), and the masks are never reused, so there is no need to spend money on the attachment/reattachment of a filter canister.

The mask with Viral-Bacterial filters welded to the mask have the modified (‘super’) filters ultrasonically welded directly onto the mask.

In the case of the oxygen masks, we place the spacer over the existing valve on the mask then weld the filter directly over it. See page 2 of Appendix 5 (Non-Rebreather Mask with Viral/Bacterial Filters) which shows the welded filter over the spacer for the oxygen mask.)

In the case of the nebulizer masks, the mask filter opening may be enlarged, and the ‘super’ filters may be ultrasonically welded over the opening. The larger mask filter opening(s) are required in order to utilize the maximum filter surface area since the aerosol saturating the filter reduces its efficiency over the procedure's duration.

Modified V/B filters may be mounted (welded) to openings on one or on both sides (left, right) of the mask, over the mask check valve, with spacer(s) disposed between the filter(s) and the check valve(s).

Text and drawings appearing in the Appendices is incorporated by reference into this specification. Some of the illustrations in the Appendices may be in the form of photographs, or other than line drawings.

FIG. 1 is a photograph of a typical facemask for oxygen/nebulizer treatments, according to the prior art. The facemask may have openings (holes) in its wall surface for allowing exhaled air to be vented from the mask. A typical facemask may have a check valve associated with the hole(s).

FIG. 2 is a diagram (cross-sectional, exploded view) of an adapter for retrofitting filters to facemasks, according to an embodiment of the invention. The adapter may have an internal component and an external component. FIG. 2 shows (from right-to-left):
An internal component which will be disposed on an interior surface of the facemask, at the location of an opening (hole) through a wall of the facemask. The internal component has a cylindrical portion with threads on an external surface thereof, and this portion extends through the wall of the facemask. A larger portion of the internal component remains on the interior of the mask, butting up against the wall of the mask on the interior surface thereof.
An external component which will be disposed on an exterior surface of the facemask. This component may be substantially cylindrical, having an internal surface which is threaded to mate with the external threads on the cylindrical portion of the internal component which is extending outside of the wall of the mask. When the external component is fitted (such as screwed) onto the internal component, the wall of the facemask is sandwiched between the internal component and the external component, with a substantially airtight seal. A flexible washer or O-ring may be provided to enhance the seal. An outer surface of the external component may be threaded to receive a filter cartridge (such as P100).
It should be understood that means other than internal threads on the external component mating with external threads on the internal component may be used for joining the internal and external components together with the facemask wall sandwiched therebetween. For example, the external surface of the internal component and internal surface of the external component may be shaped to effect a “snap” fit, or the like, including a “bayonet” type connection involving inserting and rotating. Such techniques are intended to allow for quick and easy installation of the adapter on the facemask, and also allow for easy removal of the adapter from the facemask. Alternatively, the internal and external components may glued together, but this would make removal of the adapter difficult.
The retrofit adapter disclosed herein may be attached to the mask via push and lock pins when a thread is not possible—or—the retrofit attachment may be attached via sticky tape that attaches to the mask (inside or outside surface) to create an airtight seal.
A check valve which may be incorporated into (or onto) the external component to allow exhaled air to be vented from the mask. Alternatively, the check valve may be incorporated into (or onto) the internal component.
Regarding the check valve, a typical facemask for oxygen or nebulizer treatment may already have a check valve incorporated into the mask, across the hole, to allow for the flow of exhaled air out of the mask through the hole in the wall of the mask. Such a check valve may be removed prior to installing the adapter, and in essence the check valve is moved from the mask surface to the filter attachment adapter.
A filter, such as a P100 filter cartridge, having a portion with internal threads for mating with the external threads on the external component so that the filter may be mounted to the mask, via the adapter. The filter is not a component of the adapter.

It may be desirable to either make the flexible mask conform to the retrofit device—or—to make the retrofit adapter flexible to conform to the mask shape.

FIG. 2A is a photograph of the internal component of the adapter shown in FIG. 2, inserted through a hole in a wall of the mask, according to an embodiment of the invention. A portion of the internal component of the adapter may project beyond the wall to be attached (connected, joined, mated) with the external component of the adapter. An external surface of the portion of the internal component which extends beyond the wall may be threaded (external threads).

FIG. 2B is a photograph of the external component of the adapter shown in FIG. 2, attaching to the internal component, according to an embodiment of the invention. The external component may be provided with internal threads to attach to (connect with) the external threads of the internal component. An external surface of the external component may be threaded (external threads) to accept a standard filter cartridge having internal threads.

The external component attaches to the internal component with threads, or with a “snap” (interference) fit to create a seal with the mask wall.

FIG. 2C is a photograph of a facemask with two filters mounted thereto, via the intermediary of two adapters (not visible) of the present invention.

### Some Applications for a Mask with Filter

With the Covid-19 focus on ventilators, many have been working on oxygen mask solutions with the ability to capture the patient's exhalation. These efforts have been focused on pressurized oxygen/air for breathing assistance.

Nebulizer and low-pressure oxygen treatments are extremely important for patient treatments that are not necessarily Covid-19 related. These methodologies currently utilize masks that exhale the air into the environment without means for filtration.

Two procedures that may benefit from the combination of mask with filter disclosed herein are oxygen assistance (low pressure) and nebulizer breathing treatments. This would apply to masks with filters integrated therewith, as well as to masks with filters retrofitted thereto (such as with the adapter disclosed herein). A mask with a filter incorporated therein may be used to give patients either oxygen or, more importantly give nebulized/aerosolized treatments to patients, without putting health care workers at risk.

The invention disclosed herein is intended to filter the exhalation cycle (exhalation phase of the overall breathing cycle of inhale/exhale) in the context of:
1) providing a patient with oxygen, and
2) providing a patient with nebulized or aerosolized medications,

with emphasis on the latter (delivering medications).

Notwithstanding the above, the present invention may be directed specifically and exclusively to providing nebulized or aerosolized medications to a patient, although it may also be suitable for providing oxygen to a patient.

Nebulizer and aerosol treatments typically deliver medication to the patient. Oxygen treatments may only have gas delivery without the medication. Whatever the application, it is important to protect first responders, doctors and nurses from the patient exhalation, and also to protect the patient from any contaminants in the ambient air.

A Few Words About Nebulizers.

Nebulizers are quite commonplace in hospitals and clinics these days and you'll even find quite a number of households that have one too.

Quite simply; a Nebulizer is not an oxygen delivery device, nor is it a humidifier. A Nebulizer is a drug delivery device that can dispense medication directly into the lungs in the form of an inhalable mist.

Nebulizers are used to treat various lung diseases such as: asthma, cystic fibrosis, chronic obstructive pulmonary disease (COPD), and other severe forms of lung infections and diseases.

The Nebulizer machine uses a mixture of processes involving oxygen, compressed air, and even ultrasonic power to atomize and vaporize liquid medication into small aerosol droplets, or a mist, that can be inhaled directly into the lungs.

There are three main types of electrical Nebulizers which can currently be found.
The Ultrasonic Wave Nebulizer is one of the first types of electrical Nebulizer that were designed and available on the market from 1964. An electronic oscillator creates a high frequency ultrasonic wave which causes mechanical vibrations in a piezoelectric element that breaks the liquid medication up into a fine mist. Because no air compression is used during this process, this type of Nebulizer is one of the quietest machines available.
Jet Nebulizers are one of the most commonly used machines available today. A Jet Nebulizer is also known as an atomizer because it uses compressed air to run through liquid medication at high speed, which allows it to be turned into an aerosol. Jet Nebulizers are commonly used by patients who are unable to use MDIs (metered-dose inhalers—the inhalers you usually see asthma sufferers using), or patients who require daily treatments—for whom MDIs can become very expensive. Although its big drawbacks are size, weight and noise, the Jet Nebulizer's big advantage is its low operating costs. And manufacturers are constantly improving on design and reducing overall weight and size, making the machine more portable.
The Vibrating Mesh Technology Nebulizer is one of the latest innovations in the market and it uses a laser-drilled mesh membrane which vibrates to refine the droplet size and force the liquid through, thereby creating a very fine mist. This technology allows for faster processing and thus decreases treatment times significantly. Some of the advantages of the VMT Nebulizer is that is decreases the amount of liquid waste as well as the undesired heating of the medical liquid. It is however far more expensive than any of the other types of Nebulizers which is one of its greatest drawbacks.

See https://omnisurge.co.za/what-are-nebulizers-and-how-do-they-work/In

In some examples of the invention described below, the Jet Nebulizer may be shown as an exemplary nebulizer, working in conjunction with the combination of mask and filter disclosed herein. The invention may be suitable for use with other types of nebulizers.

Providing filtration of air exhaled by the patient during these treatments is very important, as evidenced by the following: “RESPIRATORY CARE OF THE NONINTUBATED PATIENT” See https://www.uptodate.com/contents/coronavirus-disease-2019-covid-19-critical-care-issues

Low flow oxygen—For patients with COVID-19, supplemental oxygenation with a low flow system via nasal cannula is appropriate (i.e., up to 6 L/min). Although the degree of micro-organism aerosolization at low flow rates is unknown, it is reasonable to surmise that it is minimal.

Higher flows of oxygen may be administered using a simple face mask, venturi face mask, or nonrebreather mask (e.g., up to 10 to 20 L/minute), but as flow increases, the risk of dispersion also increases, augmenting the contamination of the surrounding environment and staff.

Some experts have suggested having patients who wear nasal cannula wear a droplet mask (e.g., during transport to protect spread to the surrounding environment), although data to support this are nonexistent.

Patients with higher oxygen requirements—As patients progress, higher amounts of oxygen are needed. Options at this point in non-COVID-19 patients are high-flow oxygen via nasal cannulae (HFNC) or the initiation of noninvasive ventilation (NIV). However, in patients with COVID-19, this decision is controversial and subject to ongoing debate. Despite this controversy, both modalities have been used variably. In retrospective cohorts, rates for HFNC use ranged from 14 to 63 percent while 11 to 56 percent were treated with NW. However, there are no data describing whether these modalities were successful at avoiding intubation.

Many experts advocate the avoidance of both modalities (i.e., proceeding to early intubation if escalating beyond 6 L/min with continued hypoxemia or increased work of breathing). This is predicated on an increased risk of aerosolization and high likelihood that patients who need these modalities will ultimately, rapidly deteriorate and require mechanical ventilation (e.g., within one to three days).

Nebulized medications (spontaneously breathing patients)—Nebulizers are associated with aerosolization and potentially increase the risk of SARS-CoV-2 transmission. In patients with suspected or documented COVID-19, nebulized bronchodilator therapy should be reserved for acute bronchospasm (e.g., in the setting of asthma or chronic obstructive pulmonary disease [COPD] exacerbation). Otherwise, nebulized therapy should generally be avoided, in particular for indications without a clear evidence-base; however some uses (e.g., hypertonic saline for cystic fibrosis) may need to be individualized. Metered dose inhalers (MDIs) with spacer devices should be used instead of nebulizers for management of chronic conditions (e.g., asthma or COPD controller therapy). Patients can use their own MDIs if the hospital does not have them on formulary.

If nebulized therapy is used, patients should be in an airborne infection isolation room, and healthcare workers should use contact and airborne precautions with appropriate personal protection equipment (PPE); this includes a N95 mask with goggles and face shield or equivalent (e.g., powered air-purifying respirator [PAPR] mask]) as well as gloves and gown.

All non-essential personnel should leave the room during nebulization. Some experts also suggest not re-entering the room for two to three hours following nebulizer administration.

Oxygen Therapy and Delivery Devices Oxygen therapy is commonly used on the majority of patients admitted the ambulance or hospital and ICU with respiratory distress.

There are generally three basic styles of oxygen delivery devices based on their design: low-flow, reservoir and high-flow. Oxygen systems can be divided into those indicated for low oxygen (<35%), moderate delivery (35%-60%) or high delivery (>60%) regarding the inspiratory oxygen fraction (FiO2) range.
Low-flow oxygen delivery systems consist of nasal cannula, nasal catheters and transtracheal catheters. They are designed to provide supplemental oxygen that is often less than the patient's total minute ventilation. Because the patient's minute ventilation exceeds flow, the oxygen delivered by the device will be diluted with ambient air and thus the inspired oxygen delivery is less than anticipated.
Simple Oxygen Mask or Non-Rebreather Oxygen Face Mask—Reservoir Systems Reservoir systems can gather and store oxygen during inspiration and exhalation. When patients' minute ventilation flow exceeds the device delivery flow they can draw from the reservoir anytime. To increase the oxygen concentration delivered, often a mask reservoir is utilized. The volume of the oxygen face mask is approximately 100-300 cm3 depending on size. It can deliver an FiO2 of 40-60% at 5-10 liters. The FiO2 is influenced by breath rate, tidal volume and pathology. The face masks are also great alternatives for patients with nasal irritations or epistaxis or if they are strictly mouth breathers. A simple oxygen mask should be utilized for just a few hours because of the low humidity delivered and the drying effects of the oxygen gas. This device is best used for short-term emergencies, operative procedures.

The non-rebreathing oxygen face mask should be used when an FiO2>40% is desired and for acute desaturation. It may deliver an FiO2 up to 90% at flow settings greater than 10 liters. This device is best utilized in acute cardiopulmonary emergencies where high FiO2 is necessary. It should be only used for less than four hours, secondary to inadequate humidity delivery and to variable FiO2 for patients who require a precise and high oxygen percentage.
Venturi Oxygen Mask or Aerosol Mask—High-flow Delivery

High-flow oxygen delivery systems provide a given oxygen concentration at a flow equaling or exceeding the patient's inspiratory flow demand. An exact delivered FiO2 can be achieved if the delivered flow exceeds the patient's total flow.

A Venturi mask can create high-flow enriched oxygen of a desired concentration as it mixes oxygen with room air. It produces an accurate and constant FiO2. The oxygen concentration level are typically set at 24, 28, 31, 35 and 40% respectively. The venturi mask is often employed when the clinician has a concern about CO2 retention or when respiratory drive is inconsistent. And it is often used in the COPD patient population where the risk of knocking out the patient's hypoxic drive is of concern.

An aerosol generating device can deliver FiO2 from 21 to 100% depending on the set up. The desired FiO2 is selected by adjusting an entrainment collar located on top of the aerosol container and the flow is often set at 10 LPM. There's a humidity device connected to the flow meter, and wide bore tubing connects this to the patient's mask. Wide bore tubing and the reservoir bag are placed in line to act as an oxygen reservoir to ensure that an exact high FiO2 is delivered. This device adds water content to the patient and can assist in liquefying retained secretions. This oxygen delivery option is ideal for patients with tracheotomies because it allows for inspired air to be oxygenated, humidified, and even heated if necessary. They can be hooked up to an aerosol mask, tracheotomy mask, and even a T-piece. During inhalation, an aerosol mist should be seen coming from the mask or reservoir. To ensure accurate oxygen administration via this system, an oxygen analyzer should be used. This device can be used to ensure a precise oxygen delivery and also maintain humidification of artificial airways.

See https://www.firstcaresolutions.co

In some of its embodiments, the present invention is particularly well-suited for and directed towards modifications to and uses for Simple Oxygen Masks or Non-Rebreather Oxygen Face Masks, such as described above. The mask may be referred to simply as “mask” or “face mask”.

According to some embodiments of the invention, generally, the aforementioned objects may be accomplished by either (i) retrofitting an existing mask with a filter or by (ii) integrating a filter into the mask.

Testing has shown that standard oxygen and nebulizer masks with the open exhalation holes vent contaminants into the surrounding air. The same masks, with a filter fitted thereto, was shown to have captures almost all of the vented contaminates. There is (not unexpectedly) some leakage at the mask/face interface when using the inexpensive masks, however, the particle velocities are near zero and do not spread far.

By incorporating a filter into otherwise standard oxygen and nebulizer masks, such as on the exhalation hole, exhalation of the patient is filtered to remove contaminants, thereby protecting other people (caregivers, visitors, etc.) in the vicinity of the patient being treated.

### Appendix 3 Current and Improved Nebulizer Methods

In this example, the patient interface is a mask. The mask covers the patient's mouth and nose. The mask has a vent (opening), typically disposed on its side wall, to allow ambient air in, and to allow exhaled air out. The mask also has an opening, typically at its front, to allow pressurized gas to be provided to the patient.

A compressed gas source provides air, under pressure, to a liquid reservoir, and the humidified air is provided to the mask

Some masks have check valves incorporated therein to allow (direct) inhalation only from a compressed gas/oxygen source, and cause (direct) exhalation to the environment surrounding the patient.

Page 1 shows a “Current Nebulizer Method” and the functioning of a pneumatic jet nebulizer, and also shows an “Improved Nebulizer Method”.

Page 2 shows an Improved Nebulizer Method

An improved oxygen/nebulizing method with means of filtering contaminants is disclosed herein, and incorporates some of the elements of the current nebulizing method described above, augmented by some teachings of the present invention.

By incorporating a filter on the mask, the following benefits may be obtained
ambient air inhaled by the patient may be filtered
air exhaled by the patient may be filtered
both ambient air inhaled by the patient and air exhaled by the patient may be filtered.

FIG. 3 is a diagram of a mask with filter, and nebulizer, illustrating a method of treatment, according to an embodiment of the invention.

The mask has one or more ventilation holes (“vents”) disposed on a sidewall thereof. The mask is provided with an oxygen/nebulizer connection, such as at a front portion thereof. The mask shown in FIG. 3 is comparable to the mask shown in FIG. 1.

A filter is shown, disposed on the vent. The mask may be manufactured with the filter already integrated therewith. Or, the filter may be retrofitted to the mask, such as with the adapter disclosed herein.

A nebulizer, which may for example be a pneumatic jet nebulizer, is shown connected via a patient circuit to the mask's oxygen/nebulizer connection.

A check valve (not shown, see FIG. 2) may be incorporated into the mask vent, into the filter, or into the adapter, to limit the vent's function to allowing air exhaled by the patient to exit the mask at the vent, passing through the filter into the environment, without allowing air to be inhaled by the patient through the vent. Alternatively, without a check valve, air may also be inhaled by the patient, through the filter, from the environment.

### Spacer

### Appendix 2, Face Mask Filter Retrofit—Alternative Embodiments

The goal is to either (i) to make the flexible mask conform to the retrofit device, or to make the retrofit flexible to conform to the mask shape.

Additionally, the retrofit attachment may be attached via push and lock pins when a thread is not possible. Alternatively, the retrofit attachment may be attached via sticky tape that attaches to the mask (inside or outside surface) to create an airtight seal.

The illustrations show a typical face mask for oxygen nebulizer treatments.

The two illustrations on page 1 show
an exhalation check valve (flow out only);
an inhalation check valve (flow in only); and
a connection for oxygen/nebulizer.

The two illustrations on the page 2 show that the retrofit attachment moves the check valve (exhaust) from the mask surface to the filter attachment.

### Appendix 5 Shows the Spacer

A unique spacer (the gray element in the photograph below) is disclosed for masks with directional flow valves that fits over the non-rebreather valve so the filter can be placed over the port without disrupting the valve flap.

### Appendix 5 Spacer

Page 1
Standard mask:

Inhalation and Exhalation Port into and from Environment
Non-rebreather mask:

Forces Inhalation to occur only through Medication/Oxygen Inflow Port.

Port is broken in smaller holes and a center peg

Port is covered by thin flexible rubber to form a valve that moves with air flow, thereby flapping outward during Exhalation but sealing the Port holes during Inhalation.

Page 2
Non-rebreather mask:
Non-Rebreather Mask With Viral/Bacterial (“V/B” or “B/V”) Filters

This shows an exemplary design for the spacer.

The spacer is mounted over the port with center peg acting as anchor and reference position

A Viral/Bacterial Filter mounts over spacer and port.

Reverse view shows behind the Viral/Bacterial Filter where the rubber flap is protected from filter by the spacer and free to move the with the flow

The spacer may comprise a generally flat plastic piece which is based on a disc having a center and a radius, but rather than being a complete disc, the spacer may be only a portion of a disc having at least two (three shown) arms extending radially from the center of the piece, the outer ends of the arms being shaped to snap fit over the mask valve. The spacer allows the valve to function (deflect outward) with the filter in place.

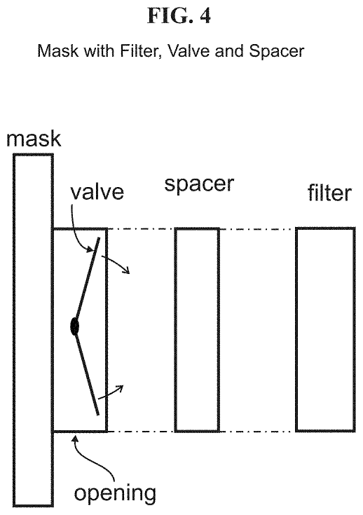

FIG. 4 illustrates (diagrammatically) a mask, fitted with a check valve (in an opening), a filter which may be disposed over the opening, and a spacer such as described above fitted over the opening between the filter and the face mask. The spacer allows the valve to function (i.e., to deflect outwards), unimpaired by the filter.

### Face Plate

A face plate, which is separate from the face mask, may be used in conjunction with the face mask to facilitate mounting the face mask to a patient's head (i.e., face).

Further modifications to the ‘retrofit mask’ have the goal of reducing aerosolization for nebulizer and oxygen breathing treatments, as well as other features, and are disclosed and described in

### Appendix 4: Face Plate Embodiment

It was determined that low cost masks leak around the facial features due to their material and cheap manufacturing.

The filters may be attached to the masks themselves (integral style) instead of making plastic cases to house the filters.

A “face plate” is provided that pushes the masks against the facial features to assure a much better fit, and also has some additional features, as may be evident from Appendix 4 (8 pages).

Page 1 Standard Mask for Nebulizing and Oxygen Treatments (Prior Art)

This page shows some views (Front/Outer, Side, Back/Inner) of a standard mask for nebulizing and oxygen treatments. Note that there open ports in the mask to permit patient breathing.

This invention may comprise some modifications to a standard mask (see, e.g., page 2), and also the addition of a face plate (see, e.g., page 3).

Page 2 Modifications to the Standard Mask to Reduce Aerosolization

This page shows a Back/Inner View of a modified mask. The following features are highlighted.

Added Nose Cushion to Increase Conformance to facial feature.

Breathing ports are covered with Viral/Bacterial filters.

Desiccant material is added to capture moisture accumulation.

Page 3 Face Plate

With a “normal” face mask, some straps are provided, extending from selected positions on the mask to features of the patient's face. Forces may be unevenly distributed about the periphery of the mask, which may allow for leakage.

In order to obtain a more air-tight seal between the mask and the patient's face, it is disclosed herein to use a separate faceplate to secure the mask to the patient's head (i.e., face). The faceplate is provided with its own straps, thereby negating the need for straps on the mask (although the mask straps may be left in place to allow first positioning the mask on the patient's face, then securing the mask to the face using the face plate/with its own straps.

The addition of a “face plate” serves to press the mask onto the facial features to improve fit.

The figure on the left illustrates that, in a conventional mask, there is leakage around nose due to stretch. A conventional mask has its own strap.

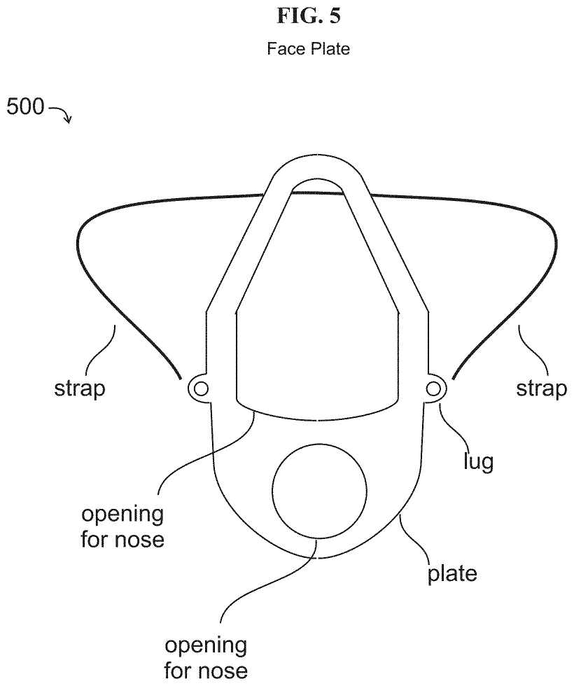

The figure on the right illustrates a faceplate which can be used with a conventional mask, or with some of the inventive mask embodiments disclosed herein. Note that the strap is relocated to the faceplate, and no longer extends from the mask itself. This provides a lot more control over fitting the mask securely to the patient's face, and may substantially reduce leakage from the mask. Compare FIG. 5

Page 4

The addition of a “face plate” serves to press the mask onto the facial features to improve fit.

The figure on the left shows the face plate on a mask. The face plate distributes the strap force onto the facial features—nose, cheeks, and chin.

The figure on the right shows that the face plate is not fixed to the mask. It is adjusted to contact the patient's nose.

Page 5

This page illustrates various ways to optimize the Face Plate to provide:

1. Filter protection from user contact

2. Strap locking features

3. Nebulizer cutout

The face plate is sized and shaped to fit over the mask, a perimeter of the face plate being generally of the same size and shape as the mask. In use, the mask is retained between the face plate and the patient's face.

The face plate has its own straps (e.g. elastic bands), such as for securing the face plate to a user's head (i.e., face) by looping the straps over the patient's ears. This eliminates the need for straps on the mask.

The face plate may be made from a more rigid material than the mask. Because the face plate is relatively more right than the mask, forces exerted by the straps (such as elastic bands) may be more evenly distributed around the periphery of the face plate, hence around the corresponding periphery of the mask, to improve sealing of the mask, as well as providing greater comfort.

The FIG. 1) on the left shows that the face plate has a cutout on the bottom of the faceplate for easy mounting of a nebulizer device.

The FIG. 2) on the right shows strap locking features on the sides of the faceplate to provide enhanced holding pressure.

Page 6

This page shows some features for optimization of the mask ‘Face Plate’ to provide:

1. Filter protection from user contact

2. Strap locking features

3. Nebulizer cutout

The sole figure shows filter protection surfaces (1. Filter protection from user contact)

Page 7 use with any nasal cannula

This page illustrates a method for using any nasal cannula End Tidal CO2 Capnography circuit with the mask.

One figure is presented, and shows:

End Tidal (ET) CO2 circuit is normally placed under the mask and into the patient's nostrils. Using a digital monitor the patient's CO2 reading is attained. When placed under the mask there is a gap between the mask and facial features thereby allowing aerosol to escape the mask.

Placing nasal cannula ports in the mask allows ET CO2 monitoring with no aerosol leakage. The nasal cannula ports are covered when not used.

A nasal cannula ports cover is shown. The cover may be printed with an instruction, such as “Remove for ET CO2”

Page 8

This page illustrates a method for using any nasal cannula End Tidal CO2 Capnography circuit with the mask. (continued from page 7)

The figures show two nasal cannula ports, a nasal cannula, and a nasal cannula installed on the mask and positioned appropriately with respect to the ports.

There have thus been disclosed and described, some modifications and/or to face masks, some methods of treatment, and a face plate for use with face masks.

While the invention(s) may have been described with respect to a limited number of embodiments, these should not be construed as limitations on the scope of the invention(s), but rather as examples of some of the embodiments of the invention(s). Those skilled in the art may envision other possible variations, modifications, and implementations that are also within the scope of the invention(s), and claims, based on the disclosure(s) set forth herein.

What is claimed is:

1. A face plate for securing a face mask to the face of a patient, comprising:
a structure having a peripheral portion conforming to a corresponding peripheral portion of the face mask, and suitable to be disposed over the mask when the mask is on the patient's face; and
at least one strap extending from the structure for securing the face plate to the patient's face, thereby negating a need for a straps extending from the face mask.

2. The face plate of claim 1, wherein:
the structure is formed of a material that is more rigid than the material of the face mask.

3. The face plate of claim 2, wherein:
the structure spreads forces from the strap(s) more evenly around the peripheral portion of the mask, thereby enhancing a seal between the mask and the patient's face.

4. The face plate of claim 1, wherein:
the structure has a cutout in an area of the patient's nose.

5. The face plate of claim 1, wherein:
the structure has a cutout in an area of the patient's mouth, for nebulizer treatment.

6. A nebulizer- or oxygen-type mask, comprising:
a bacterial/viral (B/V) filter disposed on an opening in the mask.

7. The mask of claim 6, wherein:
a peripheral portion of the B/V filter is welded to a corresponding peripheral portion of the opening in the mask.

8. The mask of claim 6, wherein:
there are two openings on the mask, one on each side (left, right) of the mask; and
there are two B/V filters, one B/V filter disposed over each of the two openings.

9. The mask of claim 6, further comprising:
a check valves disposed in the opening, for allowing a patient's exhaled air to be exhausted from within the mask, to the filter covering the opening in the mask.

10. The mask of claim 9, further comprising:
a spacer disposed between the check valve and the filter for allowing the check valve to operate (open), unimpaired by the filter.

11. The mask of claim 6, further comprising a separate face plate for securing a face mask to the face of a patient, the face plate comprising:
a structure having a peripheral portion conforming to a corresponding peripheral portion of the face mask, and suitable to be disposed over the mask when the mask is on the patient's face; and
at least one strap extending from the structure for securing the face plate to the patient's face, thereby negating a need for a straps extending from the face mask.

12. A method of providing oxygen and/or a nebulizing treatment to a patient, comprising:
providing a mask fitting over a patient's mouth and nose, said mask comprising:
(i) an opening (oxygen/nebulizer connection), typically located at the front of the mask, for admitting pressurized gas (oxygen) and/or a nebulized treatment, and
(ii) a ventilation opening (vent) on a sidewall thereof for allowing ambient air to be inhaled by the patient and also allowing air exhaled by the patient to be exhausted to the environment;
characterized by:
providing a filter on the vent.

13. The method of claim 12, further comprising:
providing an adapter for retrofitting the filter to the mask.

14. The method of claim 12, wherein:
the filter is provided “integrally” with the mask.

15. The method of claim 12, further comprising:
a check valve associated with the vent, either on the mask itself, or on the filter, or on the adapter, for allowing air exhaled by the patient to be expelled through the filter into the environment without allowing air to be inhaled by the patient through the filter and vent.

16. A face mask for treating a patient, comprising:
a check valve in an opening on the mask;
a filter disposed over the opening; and
a spacer disposed between the check valve and the filter,

17. The face mask of claim 16, further comprising:
a spacer allowing the check valve to move, and perform its intended function.

18. The face mask of claim 16, wherein:
there are two openings, each having a check valve;
there are two filters disposed over the respective two openings; and
there are two spacers disposed between the respective check valves and filters.

19. The face mask of claim 16, wherein:
the filter is securely attached to the face mask.

20. The face mask of claim 19, wherein:
the filter is ultrasonically welded to the face mask.
